(19) United States
(12) Patent Application Publication
(10) Pub. No.: US 2025/0235268 A1
(43) Pub. Date: Jul. 24, 2025

(54) ENDOSCOPE AND ASSOCIATED ROBOTIC SURGERY SYSTEM

(71) Applicant: Schölly Fiberoptic GmbH, Denzlingen (DE)

(72) Inventors: Hannes JOSEPH, Bahlingen (DE); David HENSLE, Freiburg (DE)

(73) Assignee: Schölly Fiberoptic GmbH, Denzlingen (DE)

(21) Appl. No.: 19/020,308

(22) Filed: Jan. 14, 2025

(30) Foreign Application Priority Data
Jan. 23, 2024 (DE) 102024101803.8

(51) Int. Cl. A61B 34/20; A61B 34/30; A61B 90/00

(52) U.S. Cl. CPC A61B 34/20; A61B 2034/2057; A61B 2034/301; A61B 90/361; A61B 90/37

(57) ABSTRACT

A multistep shaft system 2 for a chip-in-tip endoscope 1 is provided, such that the endoscope 1 can be precisely positioned in space by a surgery robot 11. In this case, the shaft 2 provides a small shaft diameter in a distal section 4 in order to extend the possibilities for use of this endoscope 1. Depending on the length of the entire shaft 2, it is also possible to configure an electronic relay circuit 23, preferably in a proximal section 5 of the endoscope shaft 2, in order to ensure a sufficient signal quality for the then long signal transmission path.

### CROSS-REFERENCE TO RELATED APPLICATIONS

This application claims priority from German Patent Application No. 10 2024 101 803.8, filed Jan. 23, 2024, which is incorporated herein by reference as if fully set forth.

### TECHNICAL FIELD

The invention relates to an endoscope comprising a rigid shaft and at least one image sensor for recording image data. In this case, the image sensor is arranged in a distal tip region of the endoscope. In other words, this endoscope is thus configured in the form of a chip-in-tip endoscope, comprising an image sensor together with an associated imaging optical unit, said image sensor being arranged at a distal end of an endoscope shaft of the endoscope. In such a case, the image transmission takes place electronically from the distal end of the endoscope shaft to the proximal end thereof.

The invention furthermore relates to a robotic surgery system comprising such an endoscope, and a robot having a movable arm, with the aid of which the endoscope is positionable in space.

The invention furthermore relates to a surgical arrangement comprising an endoscope according to the invention and a so-called trocar tube. This trocar tube has a working channel, the insertion opening of which is sealed by at least one seal, specifically as soon as a distal section of the shaft of the endoscope, more precisely of the endoscope shaft, is inserted into the working channel through the insertion opening. In this state, the proximal section of the endoscope remains outside the working channel.

Finally, the invention also relates to the use of a robotic surgery system, wherein the surgery system can likewise be configured according to the invention.

### BACKGROUND

Chip-in-tip endoscopes that can record 3D images are already known in the prior art. For this purpose, the imaging optical unit of the endoscope then has a stereo optical unit. Such endoscopes are typically guided manually by the surgeon and often have a shaft diameter of 4.0-5.0 mm.

### SUMMARY

Proceeding from this prior art, the invention addresses the problem of extending the possibilities for application of endoscopes in the case of use in the context of a robotic surgery system.

In order to solve this problem, one or more of the features disclosed herein are provided according to the invention in the case of an endoscope. In particular, therefore, in order to solve the problem, in the case of an endoscope of the type mentioned in the introduction, the invention proposes that the shaft is divided into a distal section and a proximal section adjacent (indirectly or directly) to the distal section, and that a minimum diameter of the proximal section of the shaft is chosen to be at least 40%, preferably even at least 60%, greater than a maximum diameter of the distal section of the shaft. As mentioned in the introduction, this endoscope should be regarded as a chip-in-tip endoscope because the image sensor is arranged wholly at the distal end of the endoscope shaft.

In other words, according to the invention, the proximal section is intended to be embodied throughout with a diameter embodied as at least 40%, preferably even at least 60%, or even at least 70%, greater than the largest diameter embodied in the distal section of the shaft. If the endoscope has for example a distal insertion region (e.g. comprising an image generating module including the at least one image sensor at the distal end of the shaft and the distal section of the shaft), the diameter of which throughout does not exceed 5.5 mm, then the above design criterion would already be satisfied with a minimum diameter of the proximal section of the shaft of at least 8.8 mm (then 45% greater).

With such a, in particular stepped, shaft or “shaft system” of the endoscope, it is thus possible to embody a chip-in-tip endoscope that can be used on a surgery robot and in this case enables on the one hand very long insertion lengths with at the same time a small shaft diameter. This is highly advantageous in laparoscopy, for example, whereas on the other hand the entire shaft system offers sufficient stiffness in order that the bending loads that occur during use can be absorbed, owing to the enlarged diameter in the proximal region.

What is advantageous about such a configuration is the possibility of enabling so-called “channel hopping”, i.e. rapid change between manipulation by a surgical tool and image generation by the endoscope during use of the same working channel/“channel” of a trocar tube for inserting the tool or else the endoscope into a patient's body during an intervention. This is made possible by the slender form of the distal section of the shaft, with the thicker proximal section ensuring the necessary mechanical stiffness of the shaft. Therefore, with the aid of a robotic surgery system (i.e. a surgical system for robot-assisted surgery), the endoscope can be reliably positioned within the working channel of the aforementioned trocar tube and/or moved therein even though the working channel is actually provided for the insertion of surgical instruments having a diameter of typically 5 mm (which constitutes a customary standard for surgical interventions). This makes it possible to significantly improve the flexibility during use of such a robotic surgery system for the surgeon, as a result of which in particular surgical interventions can be carried out more quickly and/or more precisely, which is in turn conducive to patient safety. The invention makes important contributions to all that.

Where a length is mentioned hereinafter, this relates to the respective axial length in the direction of a longitudinal direction of the shaft. By contrast, the respective diameter can be measured in a respective radial plane that is in each case perpendicular to this longitudinal axis of the shaft.

By way of example, it can be sufficient according to the invention to configure a maximum insertion length of 300 mm of the distal section of the shaft with a diameter of a maximum of 5 mm. A second shaft diameter can then be adjacent to this in a proximal direction, said second shaft diameter being significantly thicker, for example at least 8.0 mm or even at least 8.5 mm. This second section can have a length of 300 mm, such that a total shaft length of approximately 600 mm can be attained overall. Typical robotic systems currently sold on the market already use endoscope shafts having a total length of at least 500 mm.

According to the invention, the problem can also be solved by further advantageous embodiments as described below and in the claims, which are explained in detail below:

By way of example, provision can be made for the endoscope to be designed for purely electronically communicating images generated by the image sensor along the shaft. The shaft of the endoscope can thus be configured in a manner free of a relay optical unit, i.e. free of any optical unit (with the exception of the imaging optical unit that generates an image on the image sensor, provided that the image generating module mentioned is positioned in the shaft. However, this imaging optical unit is disposed upstream of the image sensor, rather than downstream thereof, in terms of the optical signal flow).

In accordance with one configuration, at least two optical channels can be formed in the distal tip region of the endoscope. In such a case, specifically, the endoscope can be designed for stereoscopic image generation with the aid of the two optical channels. This can be done in particular on the basis of two separate image sensors arranged in the distal tip region, or else on the basis of just a single image sensor then having a rectangular image sensor area of appropriate size. As an alternative to stereo image generation by two separate image sensors, just a single image sensor can thus be used as well, which then has two separate regions on a common active area of the image sensor, said area enabling respective images to be captured by sensor in order in this way likewise to enable stereoscopic image generation.

According to the invention, a minimum length of a distal insertion region of the shaft of the endoscope (said distal insertion region also comprises the distal section of the shaft that was explained in the introduction) can comprise a length of at least 180 mm, but preferably at least 200 mm. Such a minimum length of the distal insertion region ensures that it is possible to optically inspect a relevant working region with the endoscope visually within a body cavity when the endoscope is inserted by its shaft into the body cavity through a trocar tube.

By contrast, according to the invention, a length of the proximal section of the shaft of the endoscope can be at least 100 mm, but preferably at least 150 mm. Such a minimum length of the proximal section makes available a sufficient gripping region in which the endoscope shaft can be securely and reliably gripped/held by a robot.

For the use with a robotic surgery system, it is furthermore advantageous if the shaft of the endoscope has a total length of at least 450 mm, preferably of at least 500 mm. The endoscope can accordingly be configured in particular in such a way that the shaft of the endoscope can be held by a robotic surgery system at the proximal section but a free distal insertion region of the shaft with a length of at least 240 mm, preferably of at least 290 mm, can nevertheless be ensured. Such a total length ensures that firstly a free insertion region of the endoscope that is long enough for insertion into a body cavity is available, a portion of this length being required for being guided through a trocar tube. In addition, the total length ensures that the proximal section can be configured with a sufficient length such that this section can be securely gripped by a movable arm of a robot and the endoscope can thus be positioned in space securely and reliably with the aid of the robot.

For this purpose, the proximal section of the shaft can have a total length of at least 200 mm or even at least 250 mm.

An endoscope according to the invention can thus be configured with a total length of 600 mm, for example, in which case then preferably the distal section has a length of at most 310 mm and the proximal section has the rest of the remaining length (minus the length of a transition region possibly present).

Provision can furthermore be made for a maximum length of an insertion region of the shaft of the endoscope, which concomitantly comprises the distal section of the shaft, to be at most 360 mm, but preferably at most 310 mm. Such a maximum length of the distal insertion region is advantageous in order to be able to position the image sensor in the distal tip region with sufficient accuracy in space. This is because if the length of the comparatively thin distal insertion region of the endoscope is too long, there is the risk of the distal section of the shaft undergoing torsion or vibrating, both of which are detrimental to reliable image generation using the endoscope.

Furthermore, provision can supplementarily be made for a shaft diameter of the distal insertion region to be at most 6.50 mm, but preferably at most 5.80 mm.

A further advantageous configuration provides for a maximum diameter of the shaft in the distal section to be at most 6.50 mm, but preferably at most 5.80 mm.

Provision can furthermore be made for a minimum diameter of the shaft in the distal section to be at least 4.0 mm. Such geometry parameters are helpful in order to make enough installation space available for the image sensor and, secondly, in order to enable the distal section of the shaft to be inserted into a standard trocar tube with 5 mm working channel.

In the proximal section, the shaft can have a minimum diameter of at least 7.0 mm, but preferably of at least 7.50 mm.

Furthermore, provision can supplementarily or alternatively be made for a maximum diameter of the shaft in the proximal section to be at most 10.0 mm, preferably even at most 9.0 mm. One preferred configuration therefore provides for a distal electronic transmission means to be arranged in the shaft within the distal section of the shaft. Said transmission means can preferably be configured in the form of a flex PCB (flexible printed circuit board). Such a transmission means enables output signals (in particular analog output signals) of the at least one image sensor to be transmitted along the shaft in a proximal direction.

In the case of this configuration, provision can furthermore be made for a separate proximal electronic transmission means to be arranged in the shaft within the proximal section of the shaft. Said transmission means can preferably be configured in the form of a shielded coaxial cable having a plurality of respectively shielded electrical lines. Said proximal transmission means can receive the output signals of the image sensor from the distal transmission means and transmit them in a proximal direction as far as a proximal end of the shaft, in particular as digital signals. In other words, the proximal transmission means can thus perform an analog-to-digital conversion of the analog signals that it receives from the distal transmission means.

One preferred configuration provides for relay electronics to be arranged within the proximal section in the shaft of the endoscope. Said relay electronics convert the output signals communicated by the distal transmission means to the proximal transmission means, specifically in particular by an analog-to-digital conversion of the signals. It is very particularly preferred if said relay electronics comprise an amplifier which can amplify the output signals before forwarding them to the proximal transmission means. Furthermore, provision can supplementarily or alternatively be made for the relay electronics to be embodied by a rigid PCB (rigid printed circuit board). The relay electronics can solve the problem of how the longer signal transmission path required owing to the long total length of the shaft can be configured such that reliable image transmission can still be ensured in conjunction with high signal quality. In particular, this resolves the conflict of goals as to how firstly as flexible use of the endoscope as possible can be made possible with the aid of a robot and how at the same time required constraints, such as sufficient mechanical stability of the shaft and sufficiently high quality of the image transmission, can be ensured.

One configuration of the endoscope provides for the distal section and the proximal section of the shaft each to be produced by extrusion. In such a case, the two sections can have been or be connected to one another by a joining method, preferably by laser welding, either indirectly using an intermediate piece or else directly. Joining methods are specified in greater detail in the standard DIN 8593, for example. The methods relevant to the invention include, in particular: welding, soldering and adhesive bonding, each using an amorphous auxiliary material (solder, adhesive, welding flux). Such methods enable the two sections of the shaft to be non-releasably connected to one another, with a fixed connection between the sections being embodied.

The distal tip region of the endoscope can be configured in particular as a movable tip segment. In this case, the viewing direction of the image sensor arranged in the distal tip region can be varied by pivoting the tip region. In other words, a viewing angle of the endoscope can then be variable relative to a longitudinal axis of the shaft.

A further configuration provides that a rotation aid is embodied in the proximal section of the shaft, and can be used to rotate the endoscope about a longitudinal axis of the shaft. Such rotation can preferably be effected by a movable arm of a robotic surgery system, as will be described in even more specific detail below. Such a rotation aid can be embodied for example by a mechanical stop, an indentation, a toothing, or some other mechanical aid for transmitting a torque to the shaft.

In order to solve the problem, a robotic surgery system of the kind as already described in the introduction is furthermore proposed. This system is now distinguished by the fact that said movable arm of the robot holds the endoscope at the proximal section of its shaft, which can preferably be realized by a gripping or clamping mechanism. Furthermore, it is particularly preferred if the system comprises a display unit by which a live image recorded by the at least one image sensor of the endoscope can be reproduced/is displayable (in particular in a stereo view). Such a display unit can be configured for example in the form of a monitor or else in the form of VR (virtual reality) glasses.

The robot can thus have a mechanical and/or electronic interface, preferably embodied on the robot arm, which interface enables the endoscope to be mechanically and/or electronically linked into the surgery system.

In order to utilize the advantages of the invention, a surgical arrangement is furthermore proposed which, as already mentioned in the introduction, comprises an endoscope, which is configured according to the invention, and a trocar tube. When such an arrangement according to the invention is used, it is preferable if the endoscope is held at the proximal section of its shaft by a movable arm of a robotic surgery system (which can be configured in particular as explained above).

Finally, a specific use of a robotic surgery system as explained above is also proposed. In the case of this use, in order to solve the problem, provision is made for the movable arm of the robot to grip and/or hold the shaft of the endoscope in the region of the proximal section, and for a longitudinal axis of the shaft to be positioned and/or rotated in space by movement of the arm. This positioning and/or rotation of the endoscope shaft can be effected in particular while the distal section of the shaft of the endoscope is already inserted into a working channel of a trocar tube (as explained above). In other words, the endoscope can thus be inserted into the working channel of the trocar tube while the robot moves the endoscope.

A possible novel field of application for such endoscopes consists in robot-assisted surgery. Here very long endoscope shafts with a length of up to 600 mm are currently used, with a shaft diameter of 8.80 mm. Such endoscopes can be inserted into a standard trocar with 8.0 mm working channel, for instance in order to record images from inside a patient's abdominal cavity. In this case, the working channel diameter of the trocar denotes the diameter of a sealing element in the working channel, such that the slightly larger shaft can still just be inserted because the sealing element is elastic and can thus nestle sealingly against the slightly larger shaft diameter.

The comparatively large shaft diameter of more than 8 mm is required here in particular in order to ensure a sufficient mechanical stiffness of the endoscope shaft when the latter is moved in space by a movable robot arm in order to correctly orient or position the image sensor situated in the tip (and thus the viewing direction).

### BRIEF DESCRIPTION OF THE DRAWINGS

The invention will now be described in more detail on the basis of exemplary embodiments, but is not restricted to these exemplary embodiments. Further developments of the invention can be obtained from the following description of a preferred exemplary embodiment in conjunction with the general description, the claims, and the drawings.

In the following description of various preferred embodiments of the invention, elements that correspond in terms of their function are denoted by corresponding reference numerals, even in the case of a deviating design or shape.

In the drawings:

FIG. 1 shows a typical surgical situation such as may occur when a robotic surgery system according to the invention is used,

FIG. 2 shows a schematic cross-sectional view through a trocar tube, into the working channel of which the distal section of the endoscope shaft of an endoscope according to the invention is inserted, and

FIG. 3 shows a highly schematic longitudinal sectional view through an endoscope configured according to the invention.

### DETAILED DESCRIPTION

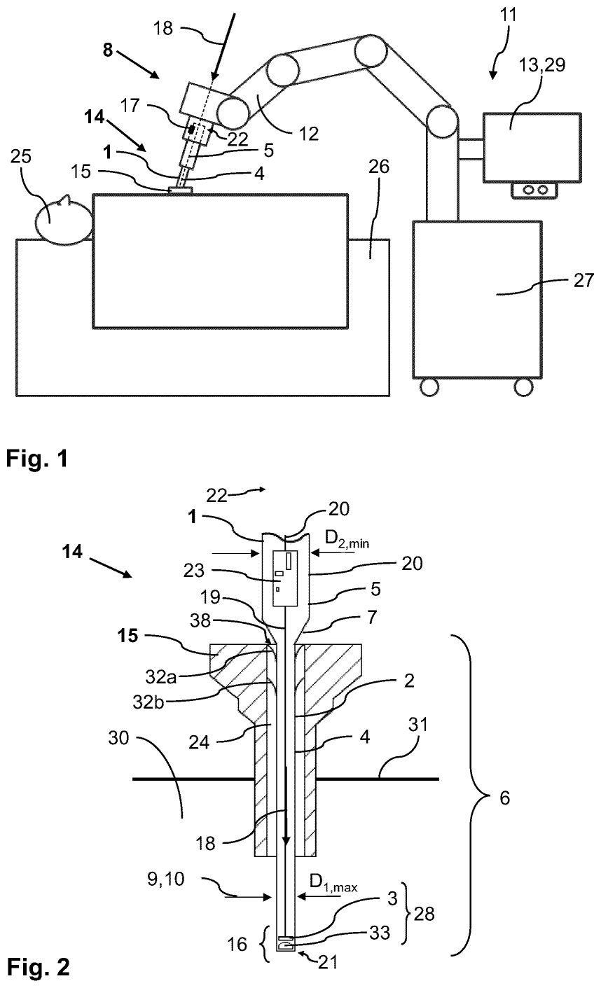

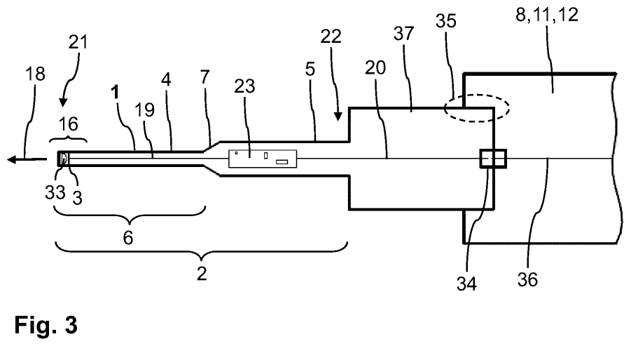

FIG. 3 shows an endoscope 1 according to the invention, which endoscope is configured as a chip-in-tip endoscope 1 and has a rigid shaft 2 comprising a distal section 4 and a proximal section 5 adjacent to the distal section 4. It is evident straightaway that an image sensor 3 with an associated imaging optical unit 33 is arranged in the distal tip region 16 at the distal end 38 of the shaft 2, the viewing direction of the image sensor 3 in this exemplary configuration running in the direction of the longitudinal axis 18 of the shaft 2 (however, an oblique view would also be configurable). It is furthermore evident that there is a handle 37 adjacent to the rigid shaft 2 at the proximal end 39 of the shaft 2. In this case, the endoscope 1 is held in the region of the handle 37 by a movable arm 12 of a robotic surgery system 8, which is also illustrated in FIG. 1.

It is furthermore noticeable in FIG. 3 that a transition region 7 is embodied between the distal section 4 and the proximal section 5, the diameter of the shaft 2 continuously increasing in a proximal direction in said transition region. In other words, the rigid endoscope shaft 2 is thus configured in a stepped manner, wherein a minimum diameter D2,min of the proximal section 5 is chosen to be more than 40% greater than a maximum diameter D1,max of the distal section 4 (in this respect, cf. FIG. 2). In the configuration shown, the diameter D1,max is both the minimum diameter 10 and the maximum diameter 9 of the shaft 2 in the distal section 4, since this section 4 is configured with a constant diameter.

Both the distal and the proximal sections 4, 5 of the shaft 2 were produced from a metal material by way of cold forming by extrusion and subsequently, by a separate metallic intermediate part forming the transition region 7, were connected to one another in a materially bonded but indirect manner by a procedure in which each of the two sections were laser-welded to the metallic intermediate part 7.

What are not shown in the figures but are likewise configurable in the context of the invention are configurations in which the endoscope shaft 2 is configured to be at least partly flexible, such that the image sensor 3 situated in the distal tip segment 16 can be actively oriented in different spatial directions, for instance in order to adapt an instantaneous field of view of the endoscope 1. A corresponding adjustment mechanism can be formed for this purpose, which mechanism is then adjustable by at least one actuating element (for example in the form of a traction cable), the actuating element then preferably being guided within the shaft 2.

In the example of an endoscope 1 according to the invention as shown in FIG. 3, the imaging optical unit 33 is embodied by a stereo optical unit, such that the endoscope 1 can be used for 3D image generation. The image sensor 3 arranged at the distal end 38 thus records different images simultaneously from different spatial directions. In this case, the images are communicated along the shaft 2 exclusively electronically by the electronic transmission means 19 and 20 illustrated in FIG. 3, which are configured in the form of a printed circuit board and a coaxial cable, respectively.

In this case, the distal electronic transmission means 19 configured as a flexible printed circuit board forwards analog output signals of the image sensor 3 firstly to relay electronics 23, which, as is readily discernible in FIG. 3, are arranged in the proximal section 5 of the shaft 2. The relay electronics 23 amplify the output signals and carry out an analog-to-digital conversion. With the aid of the proximal transmission means 20 realized by a shielded coaxial cable, these digital signals pass to an electronic interface 34 at the proximal end of the endoscope 1. Proceeding from the electronic interface 34, the digital signals are forwarded with the aid of a further transmission means 36, namely a video cable of the robotic surgery system 8, to a video system cart 27 (cf. FIG. 1), where the image signals are subjected to further image processing before finally being displayed on a monitor 29 (cf. FIG. 1).

The technical advantages of the configuration of the endoscope 1 according to the invention can be readily comprehended with reference to FIG. 2: This reveals in cross section a trocar tube 15, the distal end of which has been inserted or is inserted through the skin surface 31 of a patient 25 right into a body cavity 30. The trocar tube 15 offers a working channel 24 with two seals 32a and 32b, which are configured in an elastic fashion. In this case, the diameter of the two seals 32 is chosen to be slightly smaller than the maximum external diameter of the distal section 4 of the endoscope shaft 2. Accordingly, the two seals 32a and 32b seal the distal section 4 once the endoscope 1 has been inserted into the working channel 24 in the direction of its longitudinal axis 18 (cf. FIG. 2).

By contrast, in the situation shown in FIG. 2, the proximal section 5 of the endoscope shaft 2, said proximal section being configured with a significantly larger external diameter, is situated outside the working channel 24. It is evident that the diameter of the proximal section 5 exceeds the maximum diameter of the working channel 24, such that the shaft 2 of the endoscope can be inserted maximally with the entire distal section 4 into the working channel 24 of the trocar tube 15. In this case, the larger minimum diameter 5 D2,min of the proximal section imparts to the shaft 2 sufficient mechanical stability to ensure that the robotic surgery system 8, as shown in FIG. 1, can position the endoscope tip precisely in space and in particular can introduce it securely into the working channel 24 of the trocar tube 15.

The trocar tube 15 is likewise indicated in the schematic view in FIG. 1, said trocar tube already having been inserted into the body of the patient 25. It is furthermore evident how the movable arm 12 of the robot 11 introduces precisely the distal section 4 of the endoscope 1 into the working channel 24. In the region of the mechanical interface 35 (explained above with regard to FIG. 3) between the endoscope 1 and the robot 11, a rotation aid 17 is additionally formed on the exterior at the proximal end of the endoscope 1. In this case, the rotation aid 17 can be embodied at the proximal section of the shaft 2 or at the handle 37 shown. With the aid of the rotation aid 17, the movable arm 12 of the robot 11 can precisely rotate the endoscope 1 about its longitudinal axis 18, for instance in order to adapt the image horizon seen by the surgeon on the display unit 13.

In summary, a multistep shaft system for a chip-in-tip endoscope 1 is proposed, such that the endoscope 1 can be precisely positioned in space by a surgery robot 11. In this case, the shaft 2 provides a small shaft diameter in a distal section 4 in order to extend the possibilities for use of this endoscope 1. In this case, depending on the length of the entire shaft 2, it is also possible to configure an electronic relay circuit 23, preferably in a proximal section 5 of the endoscope shaft 2, in order to ensure a sufficient signal quality for the then long signal transmission path.

With the aid of this endoscope 1 according to the invention, firstly a very flexible use of the endoscope 1 during robotic surgical operations is made possible, in particular on account of a sufficient mechanical stability of the endoscope shaft 2. Moreover, a sufficiently high quality of the image transmission is also ensured in this way.

When the endoscope 1 is used according to the invention, therefore, only the thinner distal section 4 of the endoscope shaft 2 is inserted into the body of the patient 25, while the significantly thicker proximal section 5 of the shaft 2 remains outside the body of the patient 25, and even outside the trocar tube 15.

### LIST OF REFERENCE SIGNS

1. endoscope
2. shaft
3. image sensor (in particular as part of a compact image generating module including imaging optical unit)
4. distal section (of 2)
5. proximal section (of 3; here the endoscope can be gripped by a robot)
6. distal insertion region (of 2/1; remains free)
7. transition region (between 4 and 5)
8. robotic surgery system
9. maximum shaft diameter (of 4)
10. minimum shaft diameter (of 4)
11. robot
12. movable arm (of 11 for the positioning of 1/2/3; the arm can be moved in space by actuators in order to insert the endoscope into the patient's body at different insertion depths and/or to position the endoscope in different spatial orientations with respect to the patient)
13. display unit (e.g. in the form of a monitor or VR glasses)
14. surgical arrangement
15. trocar tube (serves for inserting 1/2/3/4 into a patient's body cavity securely and in a sealed manner; the access to the body cavity is opened by the surgeon by a trocar, and the access is kept open during the surgical operation with the aid of the associated trocar tube)
16. distal tip region (of 1/2; here is where 3 is arranged)
17. rotation aid
18. longitudinal axis
19. distal electronic transmission means (realized e.g. with the aid of a flexible printed circuit board=flex PCB)
20. proximal electronic transmission means
21. distal end (of 2)
22. proximal end (of 2)
23. relay electronics
24. working channel (of 15)
25. patient
26. operating table
27. video system cart
28. image generating module
29. monitor
30. body cavity
31. skin surface
32. seal
33. imaging optical unit
34. electronic interface (between 8/11 and 1)
35. mechanical interface (between 8/11 and 1/3)
36. transmission means (of 8, in particular video cable)
37. handle/grip
38. insertion opening (of 15)

1. An endoscope (1), comprising:
a rigid shaft (2); and
at least one image sensor (3) for recording image data, the at least one image sensor (3) being arranged in a distal tip region (16) of the endoscope (1); wherein
the shaft (2) is divided into a distal section (4) and a proximal section (5) adjacent, indirectly or directly, to the distal section (4); and
a minimum diameter of the proximal section (5) is at least 40% greater than a maximum diameter of the distal section (4).

2. The endoscope (1) as claimed in claim 1, wherein the endoscope (1) is configured for purely electronically communicating images generated by the image sensor (3) along the shaft (2), in which the shaft (2) is free of a relay optical unit.

3. The endoscope (1) as claimed in claim 1, wherein at least two optical channels are formed in the distal tip region (16) for stereoscopic image generation with the aid of the two optical channels.

4. The endoscope (1) as claimed in claim 3, wherein the at least one image sensor includes two separate image sensors (3a, 3b) arranged in the distal tip region (16), with each of the two separate image sensors (3a, 3b) being associated with a respective one of the two optical channels.

5. The endoscope (1) as claimed in claim 1, wherein a minimum length of a distal insertion region (6) of the shaft (2), which concomitantly comprises the distal section (4) of the shaft (2), is at least 180 mm.

6. The endoscope (1) as claimed in claim 1, wherein a length of the proximal section (5) of the shift (2) is at least 100 mm.

7. The endoscope (1) as claimed in claim 1, wherein the shaft (2) has a total length of at least 450 mm such that the shaft (2) is adapted to be held by a robotic surgery system (8) at the proximal section (5) and a free distal insertion region (6) of the shaft (2) with a length of at least 240 mm is ensured.

8. The endoscope (1) as claimed in claim 7, wherein a maximum length of the distal insertion region (6) of the shaft (2), which concomitantly comprises the distal section (4) of the shaft (2), is at most 360 mm, and a shaft diameter of the distal insertion region is at most 6.50 mm.

9. The endoscope (1) as claimed in claim 1, wherein a maximum diameter (9) of the shaft (2) in the distal section (4) is at most 6.50 mm.

10. The endoscope (1) as claimed in claim 1, wherein a minimum diameter (10) of the shaft (2) in the distal section (4) is at least 4.0 mm.

11. The endoscope (1) as claimed in claim 1, a minimum diameter (10) of the shaft (2) in the proximal section (4) is at least 7.0 mm.

12. The endoscope (1) as claimed in claim 1, wherein a maximum diameter (9) of the shaft (2) in the proximal section (5) is at most 10.0 mm.

13. The endoscope (1) as claimed in claim 1, further comprising a distal electronic transmission means (19) arranged in the shaft (2) within the distal section (4), the distal electronic transmission means (19) is adapted to transmit output signals of the at least one image sensor (3) along the shaft (2) in a proximal direction, and
a separate proximal transmission means (20) arranged in the shaft (2) within the proximal section (5), and the separate proximal transmission means (20) is adapted to receive the output signals from the distal transmission means (19) and transmit them in a proximal direction as far as a proximal end (22) of the shaft (2).

14. The endoscope has claimed in claim 13, further comprising relay electronics (23) arranged within the proximal section (5) in the shaft (2), wherein the relay electronics (23) are configured to convert the output signals communicated by the distal transmission means (19) to the proximal transmission means (20), and the relay electronics (23) comprise an amplifier which is adapted to amplify the output signals before forwarding them to the proximal transmission means (20).

15. The endoscope (1) as claimed in claim 1 claims, wherein the distal section (4) and the proximal section (5) are each produced by extrusion and the distal and proximal sections (4, 5) have are adapted to be connected to one another by a joining method.

16. The endoscope (1) as claimed in claim 1, wherein the distal tip region (16), in which the at least one image sensor (3) is arranged, is configured as a movable tip segment, such that a viewing angle of the endoscope (1) is variable relative to a longitudinal axis (18) of the shaft (2).

17. The endoscope (1) as claimed in claim 1, further comprising a rotation aid (17) at the proximal section (5) of the shaft (2), which is adapted to be used to rotate the endoscope (1) about a longitudinal axis (18) of the shaft (2), and the rotation aid (17) includes a mechanical stop, an indentation, a toothing, or mechanical aid for transmitting torque to the shaft (2).

18. A robotic surgery system (8), comprising:
the endoscope (1) as claimed claim 1;
a robot (11) having a movable arm (12), by which the endoscope (1) is positionable in space;
wherein the arm (12) holds the endoscope (1) at the proximal section (5) of the shaft (2); and
a display unit (13) by which a live image recorded by the at least one image sensor (3) of the endoscope (1) is adapted to be reproduced.

19. A surgical arrangement (14), comprising:
the endoscope (1) as claimed in claim 1; and
a trocar tube (15) having a working channel (24), the insertion opening (38) of which is sealed by at least one seal (32a, 32b) as soon as the distal section (4) of the shaft (2) of the endoscope is inserted into the working channel (24) through the insertion opening (38), wherein the proximal section (5) of the shaft (2) remains outside the working channel (24).

20. The use of a robotic surgery system (8) configured as claimed in claim 18, wherein
the movable arm (12) grips and/or holds the shaft (2) of the endoscope (1) in a region of the proximal section (5), and
a longitudinal axis (18) of the shaft (2) is at least one of positioned or rotated in space by movement of the arm (12).
